(19) United States
(12) Patent Application Publication
(10) Pub. No.: US 2025/0359860 A1
(43) Pub. Date: Nov. 27, 2025

(54) LEFT ATRIAL APPENDAGE CLOSURE DEVICE

(71) Applicant: BOSTON SCIENTIFIC SCIMED, INC., MAPLE GROVE, MN (US)

(72) Inventors: Jerry Sy-Shyan Wang, Edina, MN (US); Joshua David Tillman, New Brighton, MN (US); Philip Andrew Litecky, Forest Lake, MN (US); Timothy Hillukka, Monticello, MN (US); Graham Krumpelmann, Stillwater, MN (US)

(73) Assignee: BOSTON SCIENTIFIC SCIMED, INC., MAPLE GROVE, MN (US)

(21) Appl. No.: 19/215,929

(22) Filed: May 22, 2025

Related U.S. Application Data
(60) Provisional application No. 63/651,847, filed on May 24, 2024.

(51) Int. Cl. A61B 17/00

(52) U.S. Cl. CPC A61B 17/0057; A61B 2017/00367; A61B 2017/00477; A61B 2017/00632

(57) ABSTRACT

A left atrial appendage closure (LAAC) device is adapted to pull a left atrial appendage (LAA) closed on itself. The LAAC device includes a tissue engagement structure that is adapted to engage tissue within the LAA and pull the tissue inwardly when the tissue engagement structure is driven from an initial actuation level towards a final actuation level. The LAAC device includes a drive assembly that is adapted to drive the tissue engagement structure from the initial actuation level towards the final actuation level.

### CROSS-REFERENCE TO RELATED APPLICATION

This application is a continuation of U.S. Patent Application Ser. No. 63/651,847, filed May 24, 2024, entitled “LEFT ATRIAL APPENDAGE CLOSURE DEVICE”, which is incorporated by reference herein in its entirety.

### TECHNICAL FIELD

The disclosure relates generally to medical devices and more particularly to medical devices that are adapted for closing a left atrial appendage closure device.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted to pull a left atrial appendage (LAA) closed on itself. The LAAC device includes a plurality of engagement arms that extend radially outwardly from a central ring, each of the plurality of engagement arms including a free end adapted to engage tissue within an ostium of the LAA. The free end of each of the plurality of engagement arms is adapted to move proximally relative to the LAA and pull tissue of the LAA radially inward as each of the plurality of engagement arms move from an initial actuation level towards a final actuation level. The LAAC device includes a drive assembly that is adapted to drive the plurality of engagement arms from the initial actuation level towards the final actuation level.

Alternatively or additionally, the drive assembly may include a threaded core rod aligned with a longitudinal axis of the LAAC device and a threaded member that is threadedly engaged with the threaded core rod and is adapted to translate as the threaded core rod is rotated relative to the threaded member. The threaded member may be adapted to push the plurality of engagement arms towards the final actuation level as the threaded member translates.

Alternatively or additionally, the threaded member may include a threaded nut that is threadedly engaged with the threaded core rod and a nut sleeve that is secured relative to the threaded nut and is adapted to engage and push the plurality of engagement arms towards the final actuation level.

Alternatively or additionally, the nut sleeve may include a proximal facing tissue engagement feature that is adapted to hold tissue of the LAA together when the plurality of engagement arms are driven towards the final actuation level.

Alternatively or additionally, the threaded core rod may be adapted to be rotated by a delivery device.

Alternatively or additionally, the threaded core rod may include a proximal end having a releasable coupling disposed thereon.

Alternatively or additionally, the plurality of engagement arms may have an initial actuation level profile in which each of the plurality of engagement arms extend proximally from the central annular ring a short distance, then curve and extend distally.

Alternatively or additionally, the plurality of engagement arms may have an initial actuation level profile in which each of the plurality of engagement arms extend proximally from the central annular ring.

Another example may be found in a left atrial appendage closure (LAAC) device that is adapted to pull a left atrial appendage (LAA) closed on itself. The LAAC device includes a tissue engagement structure that is adapted to engage tissue within the LAA and pull the tissue inwardly when the tissue engagement structure is driven from an initial actuation level towards a final actuation level, and a drive assembly that is adapted to drive the tissue engagement structure from the initial actuation level towards the final actuation level.

Alternatively or additionally, the tissue engagement structure may include a plurality of engagement arms that each have a conjoined end and a free end. The the conjoined end of each of the plurality of engagement arms are secured together. The free end of each of the plurality of engagement arms may be adapted to engage tissue within an ostium of the LAA.

Alternatively or additionally, the free end of each of the plurality of engagement arms may be adapted to move proximally relative to the LAA as each of the plurality of engagement arms move from an initial actuation level towards a final actuation level.

Alternatively or additionally, the drive assembly may include a threaded core rod that is aligned with a longitudinal axis of the LAAC device and a threaded member that is threadedly engaged with the threaded core rod and that may be adapted to translate as the threaded core rod is rotated relative to the threaded member. The threaded member may be adapted to push the plurality of engagement arms together as the threaded member translates.

Alternatively or additionally, the threaded member may include a threaded nut that is threadedly engaged with the threaded core rod and a nut sleeve that is secured relative to the threaded nut and may be adapted to engage and push the plurality of engagement arms towards the final actuation level.

Alternatively or additionally, the plurality of engagement arms may have an initial actuation level profile in which each of the plurality of engagement arms extend proximally from the central annular ring a short distance, then curve and extend distally.

Alternatively or additionally, the plurality of engagement arms may have an initial actuation level profile in which each of the plurality of engagement arms extend proximally from the central annular ring.

Another example may be found in a left atrial appendage closure (LAAC) device that is adapted to pull a left atrial appendage (LAA) closed on itself. The LAAC device includes a tissue engagement structure that is adapted to engage tissue within the LAA and pull the tissue inwardly when the tissue engagement structure is driven from an initial actuation level towards a final actuation level, the tissue engagement structure including a plurality of engagement arms that each have a conjoined end and a free end. The conjoined end of each of the plurality of engagement arms are secured together, and the free end of each of the plurality of engagement arms are adapted to engage tissue within an ostium of the LAA. The LAAC device includes a drive assembly that is adapted to drive the plurality of engagement arms from the initial actuation level towards the final actuation level, the drive assembly including a threaded core rod aligned with a longitudinal axis of the LAAC device and a threaded member threadedly engaged with the threaded core rod and adapted to translate as the threaded core rod is rotated relative to the threaded member.

Alternatively or additionally, the threaded member may be adapted to push the plurality of engagement arms towards the final actuation level as the threaded member translates.

Alternatively or additionally, the threaded member may include a threaded nut that is threadedly engaged with the threaded core rod and a nut sleeve that is secured relative to the threaded nut and is adapted to engage and push the plurality of engagement arms towards the final actuation level.

Alternatively or additionally, the nut sleeve may include a proximal facing tissue engagement feature that is adapted to hold tissue of the LAA together when the plurality of engagement arms are driven towards the final actuation level.

Alternatively or additionally, the threaded core rod may be adapted to be releasably secured to and rotated by a delivery device.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a schematic view of an illustrative LAAC (left atrial appendage closure) device shown attached to a delivery device and disposed within an LAA;

FIG. 3 is a schematic view of the illustrative LAAC device and LAA of FIG. 2, shown after the LAAC device has been actuated to close the LAA;

FIG. 4 is a perspective view of an illustrative LAAC device;

FIG. 5 is a cross-sectional view taken along the line 5-5 of FIG. 4;

FIGS. 6A, 6B, 6C, 6D, 6E and 6F are side views of the illustrative LAAC device of FIG. 4, showing the illustrative LAAC device at first stage of actuation, a second stage of actuation, a third stage of actuation, a fourth stage of actuation, a fifth stage of actuation and a sixth stage of actuation, respectively; and

FIG. 7 is a perspective view of an illustrative LAAC device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

A left atrial appendage closure (LAAC) device that is adapted to pull a left atrial appendage (LAA) down on itself includes a plurality of engagement arms extending radially outwardly from a central ring, each of the plurality of engagement arms including a free end adapted to engage tissue within an ostium of the LAA. The free end of each of the plurality of engagement arms is adapted to move proximally relative to the LAA and pull tissue of the LAA radially inward as each of the plurality of engagement arms move from an initial actuation level towards a final actuation level. A drive assembly is adapted to drive the plurality of engagement arms from the initial actuation level towards the final actuation level.

In some cases, the drive assembly may include a threaded core rod that is aligned with a longitudinal axis of the LAAC device and a threaded member that is threadedly engaged with the threaded core rod and is adapted to translate as the threaded core rod is rotated relative to the threaded member. The threaded member is adapted to push the plurality of engagement arms towards the final actuation level as the threaded member translates. In some cases, the threaded member includes a threaded nut that is threadedly engaged with the threaded core rod and a nut sleeve that is secured relative to the threaded nut and is adapted to engage and push the plurality of engagement arms towards the final actuation level. In some cases, the nut sleeve may include a proximal facing tissue engagement feature that is adapted to hold tissue of the LAA together when the plurality of engagement arms are driven towards the final actuation level. In some cases, the threaded core rod may be adapted to be rotated by a delivery device. In some cases, the threaded core rod may include a proximal end having a releasable coupling disposed thereon.

In some cases, the plurality of engagement arms may have an initial actuation level profile in which each of the plurality of engagement arms extend proximally from the central annular ring a short distance, then curve and extend distally. This may be referred to as an inverted profile, for example. In some cases, the plurality of engagement arms may have an initial actuation level profile in which each of the plurality of engagement arms extend proximally from the central annular ring. This may be referred to as a non-inverted profile.

A left atrial appendage closure (LAAC) device adapted to pull a left atrial appendage (LAA) closed on itself includes a tissue engagement structure that is adapted to engage tissue within the LAA and pull the tissue inwardly when the tissue engagement structure is driven from an initial actuation level towards a final actuation level and a drive assembly that is adapted to drive the tissue engagement structure from the initial actuation level towards the final actuation level.

In some cases, the tissue engagement structure includes a plurality of engagement arms that each have a conjoined end and a free end, the conjoined end of each of the plurality of engagement arms secured together, and the free end of each of the plurality of engagement arms adapted to engage tissue within an ostium of the LAA. In some cases, the free end of each of the plurality of engagement arms may be adapted to move proximally relative to the LAA as each of the plurality of engagement arms move from an initial actuation level towards a final actuation level.

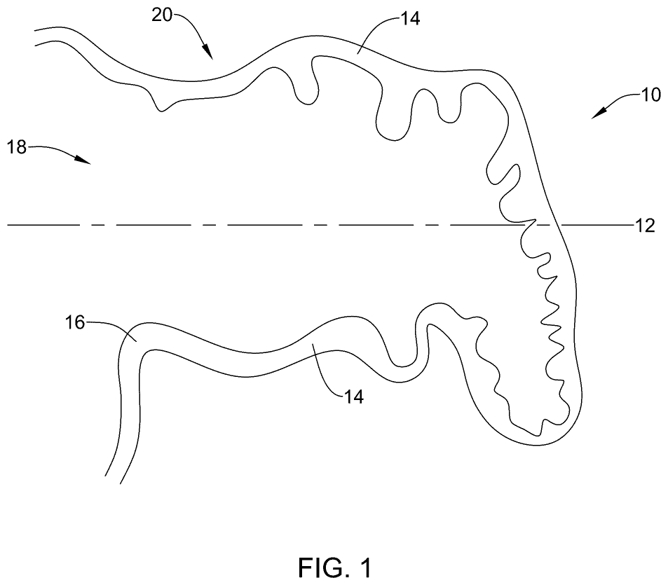

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

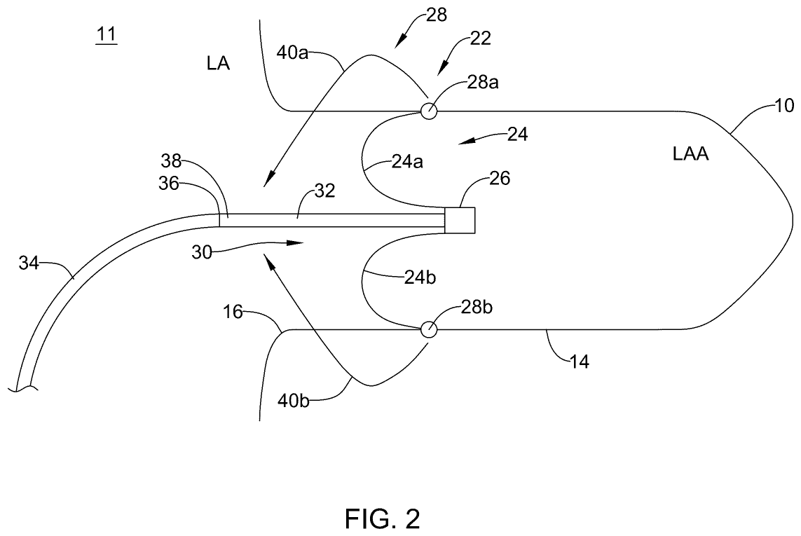

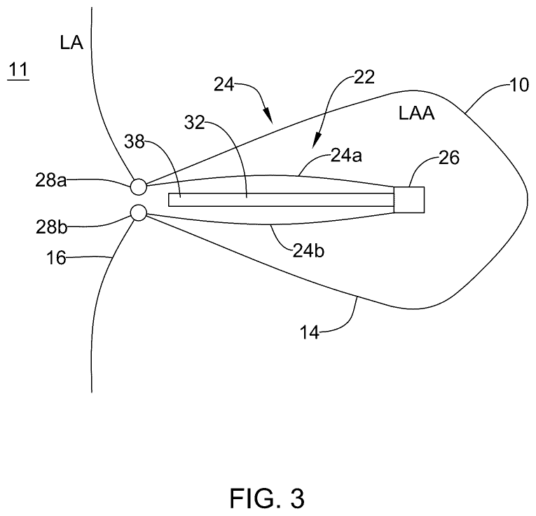

FIG. 2 is a schematic view showing the LAA 10 extending off a left atrium 11 with an illustrative LAAC (left atrial appendage closure) device 22 disposed within the LAA 10. The LAAC device 22 may be used to close the LAA 10 in order to prevent any thrombi forming within the LAA 10 from exiting the LAA 10. In some cases, the LAAC device 22 may be adapted to close up the LAA 10 by pulling the tissue of the LAA 10 down onto itself. As a result, the LAAC device 22 remains largely inside of the LAA 10 (as will be discussed with respect to FIG. 3). As a result, the LAAC device 22 is not exposed to blood flow outside of the LAA 10. In some cases, keeping the LAAC device 22 away from blood flow outside of the LAA 10 reduces or even eliminates the possibility of any issues with DRT (device-related thrombosis) that could otherwise occur if the LAAC device 22 was exposed to blood flow over a period of time. As shown in FIG. 2, the LAAC device 22 may be considered as being at an initial stage of actuation. As an example, FIG. 2 may represent a zero percent level of actuation of the LAAC device 22.

The LAAC device 22 includes a plurality of engagement arms 24 that extend radially outwardly from a central ring 26. Two engagement arms, individually labeled as 24a and 24b, are schematically shown in FIG. 2. In some cases, the LAAC device 22 may include three, four, five, six or more engagement arms 24. Each of the engagement arms 24 include a first portion that extends proximally from the central ring 26. Each of the engagement arms 24 include a second portion that curves over on itself and then extends distally. Each of the engagement arms 24 include a free end 28, individually labeled as 28a and 28b. As will be discussed, each of the free ends 28 may be adapted to anchor into the tissue of the LAA 10, such as but not including anchoring into the lateral wall 14 of the LAA 10 and/or the ostium 16 of the LAA 10.

The LAAC device 22 also includes a drive assembly 30. The drive assembly 30 includes a core drive rod 32. Some of the details of the drive assembly 30 are not visible in this view and will be discussed with respect to subsequent drawings. In some cases, the LAAC device 22 may be delivered and actuated using a delivery device 34. In some cases, the delivery device 34 may have a distal end 36 that releasably engages a proximal end 38 of the core drive rod 32 so that the delivery device 34 may be used to rotate the core drive rod 32.

Rotating the core drive rod 32 causes additional components of the drive assembly 30 (not shown in FIG. 2) to translate over the engagement arms 24 and deflect the engagement arms 24 in a direction indicated by arrows 40a and 40b, respectively. Because the free ends 28 of each of the engagement arms 24 are engaged with tissue of the LAA 10, the LAAC device 22 will cause the tissue of the LAA 10 to be pulled over itself, as shown in FIG. 3. In FIG. 3, the delivery device 34 has been withdrawn, as its use in delivering and actuating the LAAC device 22 has been completed.

FIG. 3 may be considered as representing a final actuation level for the LAAC device 22. In some cases, FIG. 3 may be considered as representing a one hundred percent level of actuation in which the LAA 10 has been pulled close, with the LAAC device 22 largely positioned inside the LAA 10 and thus isolated from blood contact with blood within the left atrium 11. In some cases, the free ends 28 of the engagement arms 24, still engaged with the tissue of the lateral wall 14 or the ostium 16 of the LAA 10, have been brought together. In some cases, the free ends 28 of the engagement arms 24 may be the only part of the LAAC device 22 that remains exposed to contact with blood within the left atrium 11.

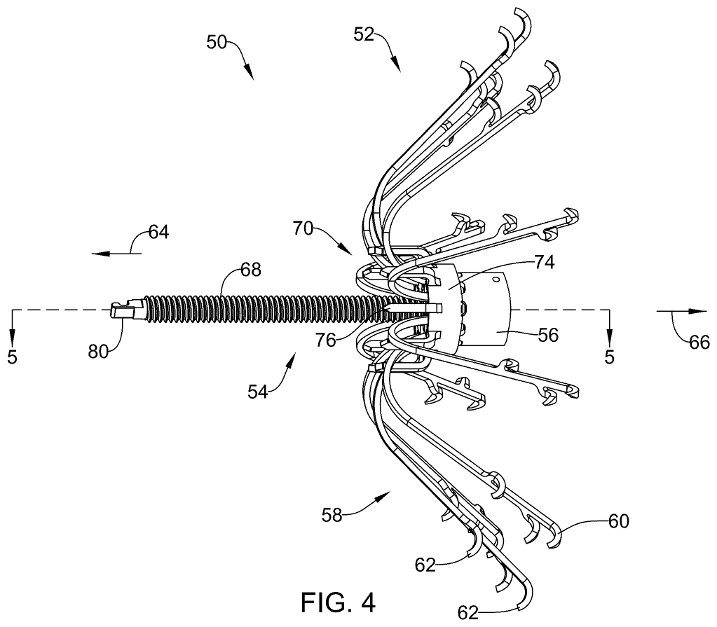

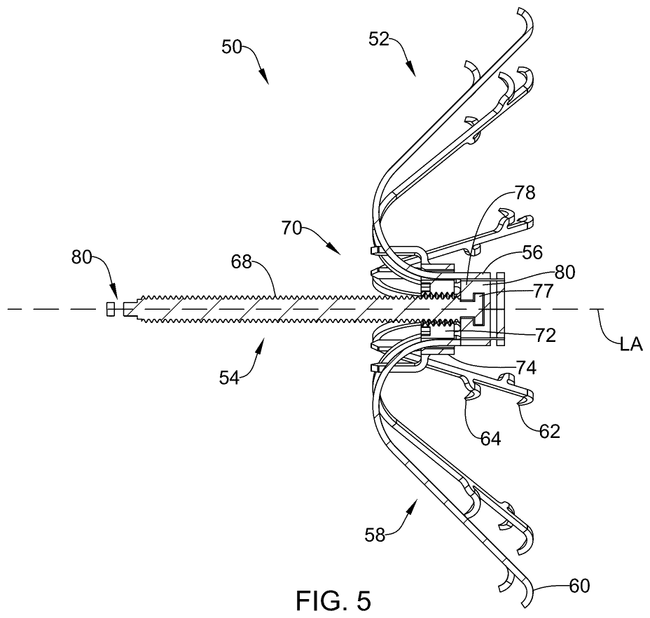

FIG. 4 is a perspective view of an illustrative left atrial appendage closure (LAAC) device 50 and FIG. 5 is a cross-sectional view thereof. The illustrative LAAC device 50 includes a tissue engagement structure 52 that is adapted to engage tissue within the LAA 10 (such as the lateral wall 14 and/or the ostium 16) and to pull the tissue inwardly when the tissue engagement structure 52 is driven from an initial actuation level towards a final actuation level. The LAAC device 50 includes a drive assembly 54 that is adapted to drive the tissue engagement structure 52 from the initial actuation level towards the final actuation level. In some cases, depending on the amount and thickness of tissue, the tissue engagement structure 52 may reach the final actuation level. In some cases, the tissue engagement structure 52 may not reach the final actuation level, with the folded layers of tissue contacting each other before the tissue engagement structure 52 has been driven to a one hundred percent actuation level.

In some cases, the tissue engagement structure 52 may include a central ring 56 and a number of engagement arms 58 that extend radially outwardly from the central ring 56. The tissue engagement structure 52 may include any number of engagement arms 58. In some cases, each of the engagement arms 58 may have the same length. In some cases, some of the engagement arms 58 may be longer than others of the engagement arms 58. Each of the engagement arms 58 may be considered as having a constrained end where the engagement arm 58 is attached to the central ring 56, and a free end 60 that is adapted to engage tissue within the lateral wall 14 and/or the ostium 16 of the LAA 10. In some instances, each of the free ends 60 may have one or more hooks or barbs 62 that facilitate the free end 60 being able to engage and hold onto tissue once the free end 60 has engaged the tissue. Each of the engagement arms 58 may include at least one hook or barb 62, and may include two or three or more hooks or barbs 62 (as shown in FIG. 4). More than three hooks or barbs 62 may also be contemplated. In some cases, each of the hooks or barbs 62 on a particular engagement arm 58 may extend in a similar radial direction. In some cases, the hooks or barbs 62 on a particular engagement arm 58 may extend in different radial directions.

In some instances, the free end 60 of each of the engagement arms 58 may be adapted to move proximally relative to the LAA 10 and pull tissue of the LAA 10 radially inward as each of the engagement arms 58 move from an initial actuation level towards a final actuation level. As an example, the tissue engagement structure 52 (both the central ring 56 and the number of engagement arms 58) may be laser-cut from a nitinol tube and may undergo one or more heat treatment processes to provide the engagement arms 58 with the profile shown in FIG. 4. In some cases, the profile shown in FIG. 4 may be considered as being an inverse profile in which each of the engagement arms 58 first extend in a proximal direction as indicated by an arrow 64 before curving back and extending in a distal direction as indicated by an arrow 66.

The drive assembly 54 includes a threaded core rod 68 that is aligned with a longitudinal axis LA of the LAAC device 50. As shown, the threaded core rod 68 has an outer threaded surface. A threaded member 70 is threadedly engaged with the threaded core rod 68 such that the threaded member 70 is able to translate relative to the threaded core rod 68 as the threaded core rod 68 is rotated relative to the threaded member 70. The threaded member 70 may be adapted to push the engagement arms 58 towards the final actuation level as the threaded member 70 translates along the threaded core rod 68.

In some cases, the threaded member 70 may include a threaded nut 72 (shown in FIG. 5) that is threadedly engaged with the threaded core rod 68 and a nut sleeve 74 that is secured relative to the threaded nut 72. The nut sleeve 74 may be adapted to engage and push the engagement arms 58 towards the final actuation level. As the threaded core rod 68 rotates, and the tissue engagement structure 52 is held from rotation via tissue engagement, the threaded nut 72 will translate along the threaded core rod 68. The nut sleeve 74 is secured relative to the threaded nut 72 and thus translates along with the threaded nut 72. As the nut sleeve 74 translates, the nut sleeve 74 causes each of the engagement arms 58 to move in the proximal direction, as indicated by the arrow 64. In some cases, the nut sleeve 74 may include a proximal facing tissue engagement feature 76 that is adapted to hold tissue of the LAA 10 together when the engagement arms 58 are driven towards the final actuation level. The proximal facing tissue engagement feature 76 may include straight barbs intended to penetrate tissue of the LAA 10 and to help hold the tissue of the LAA 10 together when the LAAC device 50 is in, or approaches, its final actuation level. The LAAC device 50 may include any number of straight proximal facing tissue engagement features 76. As an example, the LAAC device 60 may have one or more straight proximal facing tissue engagement features 76 and as many as ten or twelve or more straight proximal facing tissue engagement features 76.

The threaded core rod 68 includes a flange 77 at or near its distal end that facilitates securing the threaded core rod 68 within a drive hard stop 78. In some cases, the threaded core rod 68 may include a releasable coupling member 80 disposed at or near its proximal end. In some cases, the releasable coupling member 80 allows the threaded core rod 68 to be releasably secured to the delivery device 34 so that rotation of the delivery device 34 causes a corresponding rotation of the threaded core rod 68. The distal end 36 of the delivery device 34 may include a releasable coupling member that is complementary to the releasable coupling member 80. As an example, the releasable coupling member 80 and the complementary releasable coupling member (not shown) at the distal end of the delivery device 34 may together form a train-coupler style coupling that may be held together via a pin extending through both members, or a sleeve extending over both members. Retracting the pin or the sleeve can subsequently allow the delivery device 34 to be removed from the LAAC device 50.

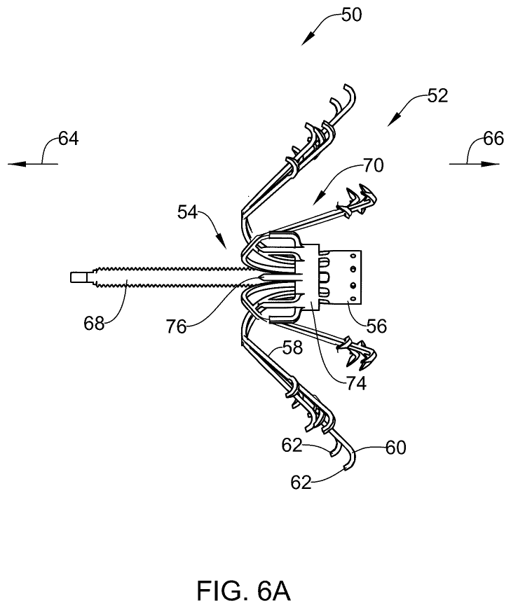

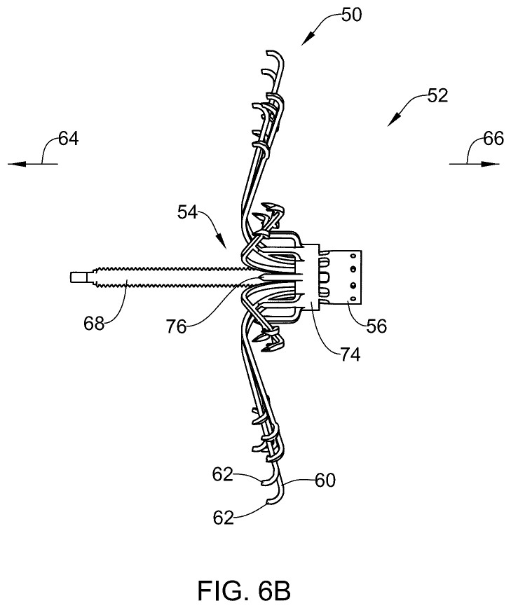

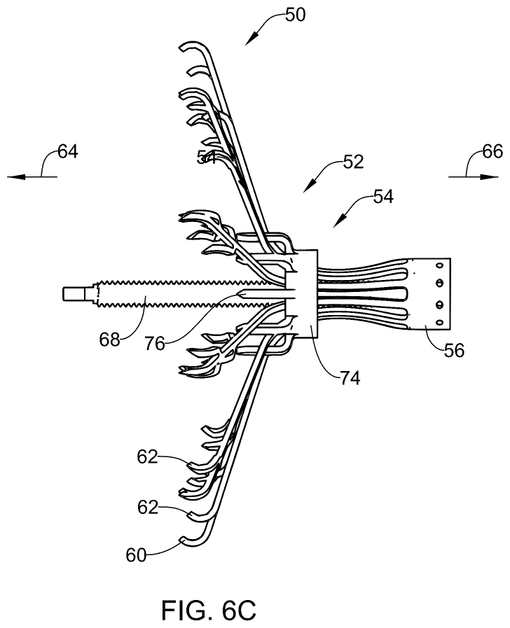

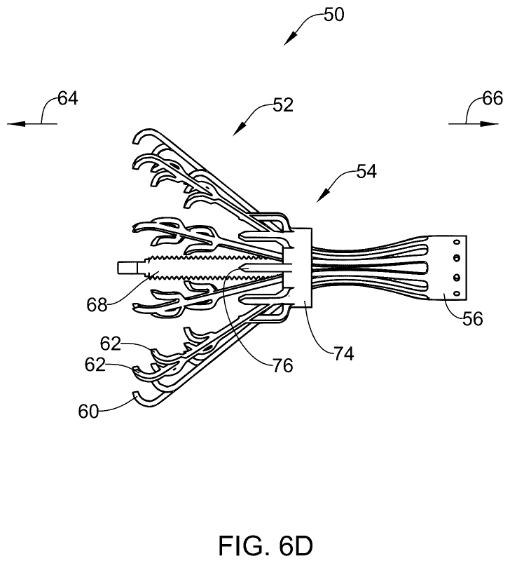

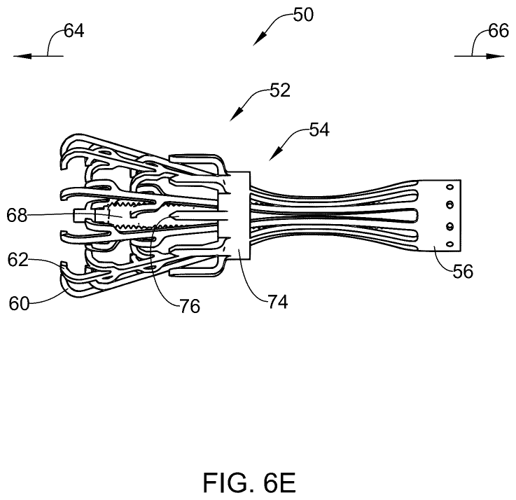

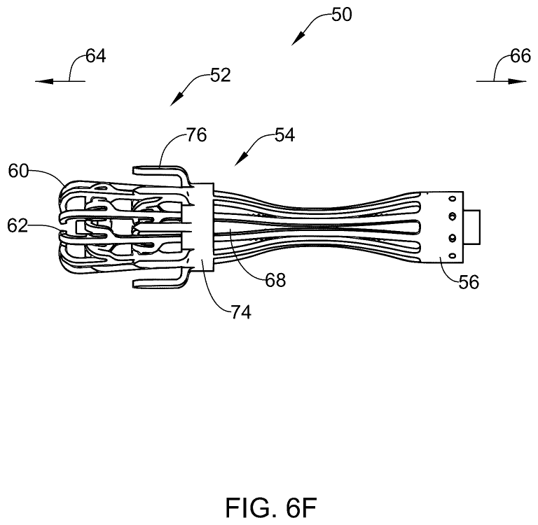

FIGS. 6A, 6B, 6C, 6D, 6E and 6F are side views of the illustrative LAAC device 50 FIG. 4, showing the LAAC device at a variety of different actuation levels. In FIG. 6A, the tissue engagement structure 52 is shown at a first stage of actuation. As an example, the first stage of actuation may correspond to about zero percent actuation, meaning that the threaded nut 72 has not moved away from the drive hard stop 78. This may correspond to when the LAAC device 50 has been initially disposed within the ostium 16 of the LAA 10. The free ends 60 of the engagement arms 58 extend axially in a distal direction as indicated by the arrow 66. The first stage of actuation may correspond to an initial actuation level, for example. In FIG. 6B, the tissue engagement structure 52 is shown at a second stage of actuation, meaning that the threaded member 70 has begun to translate as a result of the threaded core rod 68 being rotated. The free ends 60 of the engagement arms 58 still extend axially in a distal direction as indicated by the arrow 66. As an example, the second stage of actuation may correspond to the threaded nut 72 having translated about twenty percent of its overall travel limit.

In FIG. 6C, the tissue engagement structure 52 is shown at a third stage of actuation, meaning that the threaded member 70 has translated further as a result of the threaded core rod 68 being rotated. The nut sleeve 74 has pushed the engagement arms 58 sufficiently far that the free ends 60 of the engagement arms 58 extend axially in a proximal direction as indicated by the arrow 64. As an example, the third stage of actuation may correspond to the threaded nut 72 having translated about forty percent of its overall travel limit. In FIG. 6D, the tissue engagement structure 52 is shown at a fourth stage of actuation, meaning that the threaded member 70 has translated further as a result of the threaded core rod 68 being rotated. It will be appreciated that an overall periphery of the tissue engagement structure 52, as indicated by the free ends 60 of each of the engagement arms 58, is reduced relative to FIG. 6C, meaning that the tissue of the LAA 10 is being pulled together. As an example, the fourth stage of actuation may correspond to the threaded nut 72 having translated about sixty percent of its overall travel limit.

In FIG. 6E, the tissue engagement structure 52 is shown at a fifth stage of actuation. It can be seen that that an overall periphery of the tissue engagement structure 52, as indicated by the free ends 60 of each of the engagement arms 58, is reduced relative to FIG. 6D, meaning that the tissue of the LAA 10 is being pulled closer together. As an example, the fifth stage of actuation may correspond to the threaded nut 72 having translated about eighty percent of its overall travel limit. In FIG. 6F, the tissue engagement structure 52 is shown at sixth stage of actuation. It can be seen that an overall periphery of the tissue engagement structure 52, as indicated by the free ends 60 of each of the engagement arms 58, is reduced relative to FIG. 6E, meaning that the tissue of the LAA 10 is being pulled closer together as much as possible. As an example, the sixth stage of actuation may correspond to the threaded nut 72 having translated about one hundred percent of its overall travel limit, meaning that the threaded nut 72 has moved as far proximally as it is able to do so. The sixth stage of actuation may be considered as corresponding to a final actuation level.

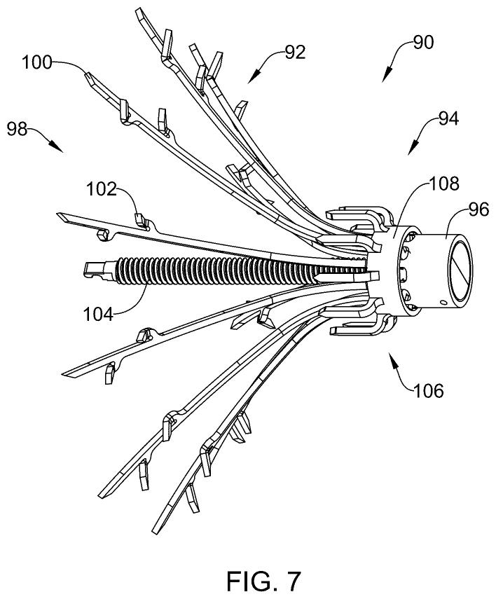

FIG. 7 is a perspective view of an illustrative left atrial appendage closure (LAAC) device 90. The illustrative LAAC device 90 includes a tissue engagement structure 92 that is adapted to engage tissue within the LAA 10 (such as the lateral wall 14 and/or the ostium 16) and to pull the tissue inwardly when the tissue engagement structure 92 is driven from an initial actuation level towards a final actuation level. The LAAC device 90 includes a drive assembly 94 that is adapted to drive the tissue engagement structure 94 from the initial actuation level towards the final actuation level. In some cases, depending on the amount and thickness of tissue, the tissue engagement structure 92 may reach the final actuation level. In some cases, the tissue engagement structure 92 may not reach the final actuation level, with the folded layers of tissue contacting each other before the tissue engagement structure 92 has been driven to a one hundred percent actuation level.

In some cases, the tissue engagement structure 92 may include a central ring 96 and a number of engagement arms 98 that extend radially outwardly from the central ring 96. The tissue engagement structure 92 may have any number of engagement arms 98. In some cases, each of the engagement arms 98 may be the same length. In some cases, some of the engagement arms 98 may be longer than other engagement arms 98. Each of the engagement arms 98 have a free end 100 that is adapted to engage tissue within the lateral wall 14 and/or the ostium 16 of the LAA 10. In some instances, each of the free ends 100 may have one or more hooks or barbs 102 that facilitate the free end 100 being able to engage and hold onto tissue once the free end 100 has engaged the tissue, and may include two or three or more hooks or barbs 102. More than three hooks or barbs 102 may also be contemplated. In some cases, each of the hooks or barbs 102 on a particular engagement arm 98 may extend in a similar radial direction. In some cases, the hooks or barbs 102 on a particular engagement arm 98 may extend in different radial directions. In some instances, the free end 100 of each of the engagement arms 98 may be adapted to move proximally relative to the LAA 10 and pull tissue of the LAA 10 radially inward as each of the engagement arms 98 move from an initial actuation level towards a final actuation level. As an example, the tissue engagement structure 92 (both the central ring 96 and the number of engagement arms 98) may be laser-cut from a nitinol tube and may undergo one or more heat treatment processes to provide the engagement arms 98 with the profile shown in FIG. 7. In some cases, the profile shown in FIG. 7 may be considered as being a non-inverse profile in which each of the engagement arms 98 extend in a proximal direction.

The drive assembly 94 includes a threaded core rod 104. As shown, the threaded core rod 104 has an outer threaded surface. A threaded member 106 is threadedly engaged with the threaded core rod 104 such that the threaded member 106 is able to translate relative to the threaded core rod 104 as the threaded core rod 104 is rotated relative to the threaded member 106. The threaded member 106 may be adapted to push the engagement arms 98 towards the final actuation level as the threaded member 106 translates along the threaded core rod 104. Similar to the threaded member 70, the threaded member 106 may include a threaded nut (not visible in FIG. 7) that is threadedly engaged with the threaded core rod 104 and a nut sleeve 108 that is secured relative to the threaded nut. The nut sleeve 108 may be adapted to engage and push the engagement arms 98 towards the final actuation level.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear-elastic” or “non-super-elastic” which, although may be similar in chemistry to conventional shape memory and super clastic varieties, may exhibit distinct and useful mechanical properties. Linear elastic and/or non-super-elastic nitinol may be distinguished from super-clastic nitinol in that the linear elastic and/or non-super-clastic nitinol does not display a substantial “super-elastic plateau” or “flag region” in its stress/strain curve like super-clastic nitinol does. Instead, in the linear-clastic and/or non-super-clastic nitinol, as recoverable strain increases, the stress continues to increase in a substantially linear, or a somewhat, but not necessarily entirely linear relationship until plastic deformation begins or at least in a relationship that is more linear that the super-elastic plateau and/or flag region that may be seen with super-elastic nitinol. Thus, for the purposes of this disclosure linear-clastic and/or non-super-elastic nitinol may also be termed “substantially” linear-elastic and/or non-super-elastic nitinol.

In some cases, linear-elastic and/or non-super-elastic nitinol may also be distinguishable from super-clastic nitinol in that linear-clastic and/or non-super-elastic nitinol may accept up to about 2-5% strain while remaining substantially elastic (e.g., before plastically deforming) whereas super-clastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear-elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (C) to about 120° C. in the linear-elastic and/or non-super-elastic nickel-titanium alloy. The mechanical bending properties of such material may therefore be generally inert to the effect of temperature over this very broad range of temperature. In some embodiments, the mechanical bending properties of the linear-elastic and/or non-super-clastic nickel-titanium alloy at ambient or room temperature are substantially the same as the mechanical properties at body temperature, for example, in that they do not display a super-clastic plateau and/or flag region. In other words, across a broad temperature range, the linear-elastic and/or non-super-clastic nickel-titanium alloy maintains its linear-clastic and/or non-super-elastic characteristics and/or properties.

In some embodiments, the linear-elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted to pull a left atrial appendage (LAA) closed on itself, the LAAC device comprising:
a plurality of engagement arms extending radially outwardly from a central ring, each of the plurality of engagement arms including a free end adapted to engage tissue within an ostium of the LAA;
the free end of each of the plurality of engagement arms adapted to move proximally relative to the LAA and pull tissue of the LAA radially inward as each of the plurality of engagement arms move from an initial actuation level towards a final actuation level; and
a drive assembly adapted to drive the plurality of engagement arms from the initial actuation level towards the final actuation level.

2. The LAAC device of claim 1, wherein the drive assembly comprises:
a threaded core rod aligned with a longitudinal axis of the LAAC device; and
a threaded member threadedly engaged with the threaded core rod, the threaded member adapted to translate as the threaded core rod is rotated relative to the threaded member;
wherein the threaded member is adapted to push the plurality of engagement arms towards the final actuation level as the threaded member translates.

3. The LAAC device of claim 2, wherein the threaded member comprises:
a threaded nut threadedly engaged with the threaded core rod; and
a nut sleeve secured relative to the threaded nut, the nut sleeve adapted to engage and push the plurality of engagement arms towards the final actuation level.

4. The LAAC device of claim 3, wherein the nut sleeve comprises a proximal facing tissue engagement feature that is adapted to hold tissue of the LAA together when the plurality of engagement arms are driven towards the final actuation level.

5. The LAAC device of claim 2, wherein the threaded core rod is adapted to be rotated by a delivery device.

6. The LAAC device of claim 5, wherein the threaded core rod includes a proximal end having a releasable coupling disposed thereon.

7. The LAAC device of claim 1, wherein the plurality of engagement arms have an initial actuation level profile in which each of the plurality of engagement arms extend proximally from the central annular ring a short distance, then curve and extend distally.

8. The LAAC device of claim 1, wherein the plurality of engagement arms have an initial actuation level profile in which each of the plurality of engagement arms extend proximally from the central annular ring.

9. A left atrial appendage closure (LAAC) device adapted to pull a left atrial appendage (LAA) closed on itself, the LAAC device comprising:
a tissue engagement structure adapted to engage tissue within the LAA and pull the tissue inwardly when the tissue engagement structure is driven from an initial actuation level towards a final actuation level; and
a drive assembly adapted to drive the tissue engagement structure from the initial actuation level towards the final actuation level.

10. The LAAC device of claim 9, wherein the tissue engagement structure comprises a plurality of engagement arms that each have a conjoined end and a free end, the conjoined end of each of the plurality of engagement arms secured together, and the free end of each of the plurality of engagement arms adapted to engage tissue within an ostium of the LAA.

11. The LAAC device of claim 10, wherein the free end of each of the plurality of engagement arms is adapted to move proximally relative to the LAA as each of the plurality of engagement arms move from an initial actuation level towards a final actuation level.

12. The LAAC device of claim 9, wherein the drive assembly comprises:
a threaded core rod aligned with a longitudinal axis of the LAAC device; and
a threaded member threadedly engaged with the threaded core rod, the threaded member adapted to translate as the threaded core rod is rotated relative to the threaded member;
wherein the threaded member is adapted to push the plurality of engagement arms together as the threaded member translates.

13. The LAAC device of claim 12, wherein the threaded member comprises:
a threaded nut threadedly engaged with the threaded core rod; and
a nut sleeve secured relative to the threaded nut, the nut sleeve adapted to engage and push the plurality of engagement arms towards the final actuation level.

14. The LAAC device of claim 10, wherein the plurality of engagement arms have an initial actuation level profile in which each of the plurality of engagement arms extend proximally from the central annular ring a short distance, then curve and extend distally.

15. The LAAC device of claim 10, wherein the plurality of engagement arms have an initial actuation level profile in which each of the plurality of engagement arms extend proximally from the central annular ring.

16. A left atrial appendage closure (LAAC) device adapted to pull a left atrial appendage (LAA) closed on itself, the LAAC device comprising:
a tissue engagement structure adapted to engage tissue within the LAA and pull the tissue inwardly when the tissue engagement structure is driven from an initial actuation level towards a final actuation level, the tissue engagement structure including a plurality of engagement arms that each have a conjoined end and a free end, the conjoined end of each of the plurality of engagement arms secured together, and the free end of each of the plurality of engagement arms adapted to engage tissue within an ostium of the LAA; and
a drive assembly adapted to drive the plurality of engagement arms from the initial actuation level towards the final actuation level, the drive assembly including a threaded core rod aligned with a longitudinal axis of the LAAC device and a threaded member threadedly engaged with the threaded core rod and adapted to translate as the threaded core rod is rotated relative to the threaded member.

17. The LAAC device of claim 16, wherein the threaded member is adapted to push the plurality of engagement arms towards the final actuation level as the threaded member translates.

18. The LAAC device of claim 16, wherein the threaded member comprises:
a threaded nut threadedly engaged with the threaded core rod; and
a nut sleeve secured relative to the threaded nut, the nut sleeve adapted to engage and push the plurality of engagement arms towards the final actuation level.

19. The LAAC device of claim 18, wherein the nut sleeve comprises a proximal facing tissue engagement feature that is adapted to hold tissue of the LAA together when the plurality of engagement arms are driven towards the final actuation level.

20. The LAAC device of claim 16, wherein the threaded core rod is adapted to be releasably secured to and rotated by a delivery device.
